(19) United States
(12) Patent Application Publication
(10) Pub. No.: US 2015/0190117 A1
(43) Pub. Date: Jul. 9, 2015

(54) ULTRASOUND MEASUREMENT INSTRUMENT AND ULTRASOUND MEASUREMENT DEVICE

(71) Applicant: FURUNO ELECTRIC CO., LTD., Nishinomiya City (JP)

(72) Inventors: Tatsuo Arai, Nishinomiya City (JP); Wataru Kiyan, Nishinomiya City (JP)

(21) Appl. No.: 14/415,301

(22) PCT Filed: Jun. 11, 2013
(86) PCT No.: PCT/JP2013/066048

§ 371 Date: Jan. 16, 2015

(30) Foreign Application Priority Data
Jul. 17, 2012 (JP) 2012-158461

(51) Int. Cl. A61B 8/00; A61B 8/08

(52) U.S. Cl. CPC A61B 8/4455; A61B 8/4272; A61B 8/4461; A61B 8/0858

(57) ABSTRACT

The ultrasound measurement instrument of the present invention comprises a main body component having a polyhedral shape; a nozzle that is provided in one plane of the main body component, the nozzle and communicating with the main body component via a hollow portion; and a contact component blocking off a face of the nozzle on the opposite side from the main body component in a flat shape such that ultrasonic waves are transmitted. The contact component is inclined with respect to a first axis that is parallel to the plane of the polyhedral body in which the nozzle is provided, and to a second axis that is perpendicular to the first axis and is parallel to the plane. A cartilage surface is inclined with respect to a biological surface, and the contact component is inclined by the same degree in the inclination direction thereof. Therefore, the ultrasonic waves emitted perpendicular to the face of the main body component where the nozzle is located pass through the contact component and proceed perpendicular to the cartilage surface.

### CROSS REFERENCE TO RELATED APPLICATIONS

This application is a U.S. National stage of International Application No. PCT/JP2013/066048 filed on Jun. 11, 2013. This application claims priority to Japanese Patent Application No. 2012-158461 filed on Jul. 17, 2012. The entire disclosure of Japanese Patent Application No. 2012-158461 is hereby incorporated herein by reference.

### BACKGROUND

1. Field of the Invention

The present invention relates to a device for using ultrasonic waves to measure the state of cartilage in a joint.

2. Background Information

In the past, to measure the state of cartilage, the user inserted an endoscope into a joint cavity for direct observation, or a non-invasive measurement method was used that featured the use of MRI or ultrasound (see International Publication No. 2008/108054 (Patent Literature 1)).

In measurement with ultrasound, an ultrasound probe that transmits and receives ultrasonic waves is used. An ultrasound probe of a measurement device emits ultrasonic waves, and receives ultrasonic waves reflected at the boundary between cartilage and soft tissue, which have different acoustic impedance. A signal processing unit of the measurement device calculates the distance from the outer skin to the cartilage surface, or the degree of modification of the cartilage, on the basis of the echo received by the ultrasound probe.

### SUMMARY

To receive reflected waves of large amplitude with an ultrasound probe, it is preferable for the ultrasonic waves to move perpendicular to the cartilage surface. However, biological surfaces are not always parallel to the surface of the cartilage to be measured. Therefore, the user has to adjust the position of the probe by trial and error so that the reflected ultrasonic waves can be received.

In view of this, it is an object of the present invention to provide an ultrasound measurement instrument and an ultrasound measurement device with which ultrasonic waves can advance perpendicular to the cartilage surface even though no complicated position adjustment is made.

The ultrasound measurement instrument of the present invention comprises a main body component having a hollow body; a nozzle provided in one plane of the main body component, the nozzle having a hollow shape and communicating with the main body component via a hollow portion; and a contact component blocking off a face of the nozzle on the opposite side from the main body component in a flat shape such that ultrasonic waves are transmitted.

The contact component is inclined with respect to a first axis that is parallel to the plane of the hollow body in which the nozzle is provided, and to a second axis that is perpendicular to the first axis and is parallel to the plane.

The ultrasound measurement instrument of the present invention comes into contact with a biological surface via the contact component when the user measures the state of cartilage with ultrasound. The ultrasonic waves are emitted from an ultrasound probe that is used integrally with the ultrasound measurement instrument, pass through the contact component, and reach the biological surface. The ultrasonic waves that reach the biological surface are reflected at the surface of soft tissue, cartilage, or bone, and some of these reflected waves pass through the contact component.

The main body component is not limited to having a hollow body, and may have a polyhedral shape or hollow sphere shape.

The cartilage surface is inclined with respect to the biological surface, but the contact component is inclined with respect to the face of the main body component where the nozzle is provided, in the same direction by the same degree as the inclination direction thereof. Therefore, ultrasonic waves that are emitted perpendicular to the face of the main body component where the nozzle is located advance in a direction that is perpendicular to the cartilage surface. The waves that are reflected by the cartilage surface advance perpendicularly from the cartilage surface and pass through the contact component. Since the reflected waves that pass through the contact component thus advance in a direction that is perpendicular to the cartilage surface, they arrive at the ultrasound probe in the shortest distance from the cartilage surface.

Also, the ultrasound measurement instrument of the present invention has an elastic body between the contact component and a portion of the nozzle that touches the contact component.

The elastic body is deformed when the ultrasound measurement instrument is pressed against a biological surface. Therefore, even if the angle at which the contact component is inclined with respect to the face of the main body component where the nozzle is located is different from the angle at which the biological surface is inclined with respect to the cartilage surface, the elastic body will deform so that the angle can be finely adjusted. The user can adjust the angle at which the contact component is inclined with respect to the face of the main body component where the nozzle is located, and the ultrasonic waves emitted perpendicular to the cartilage surface. Also, when the elastic body of the ultrasound measurement instrument deforms, the local pressure exerted on the biological surface is dispersed, which reduces the damage to the biological surface.

Silicone rubber, for example, is used for the elastic body.

The contact component can have a shape that combines a straight part and a curved part. In this case, because the contact component does not have a pointed shape, there is less damage to the biological surface during pressing.

A silicone film, for example, is used for the contact component.

Also, the nozzle of the ultrasound measurement instrument of the present invention can have a column shape that is constricted toward a face that is opposite a face of the main body component where the nozzle is provided.

Also, the ultrasound measurement device of the present invention comprises the above-mentioned ultrasound measurement instrument, an ultrasound probe configured to transmit and receive ultrasonic waves that pass through the contact component, and a drive mechanism configured to drive the ultrasound probe in an interior of the main body component. The ultrasound probe emits ultrasonic waves that advance perpendicular to the face of the main body component where the nozzle is located. The drive mechanism moves the ultrasound probe parallel to the face of the main body component where the nozzle is located. Therefore, the user can send an instruction to the drive mechanism and thereby move the measurement location and continuously measure the state of cartilage.

The drive mechanism of the ultrasound measurement device of the present invention preferably moves the ultrasound probe parallel to a face of the main body component where the nozzle is provided. Because of this configuration of the present invention, scanning can be performed successively, so that the ultrasonic waves are incident perpendicular to the knee joint cartilage.

Also, the ultrasound measurement method of the present invention comprises a step in which the above-mentioned ultrasound measurement device emits ultrasonic waves perpendicular to a face of the main body component where the nozzle is provided, and a step of receiving ultrasonic waves that have been reflected by knee joint cartilage and have passed through the contact component.

Thus, with the ultrasound measurement instrument of the present invention, ultrasonic waves can advance perpendicular to the cartilage surface even though no complicated position adjustment is made.

### BRIEF DESCRIPTION OF THE DRAWINGS

FIG. 1 is an oblique view of the exterior of an ultrasound measurement device.

FIG. 2 is a side view of an ultrasound measurement device.

FIG. 3 is a diagram of the ultrasound measurement device as seen from the opposite side from that in FIG. 2.

FIG. 4 is a bottom view of an ultrasound measurement device.

FIGS. 5A and 5B are side views of the interior of a bent right knee joint.

FIGS. 6A and 6B are side views of the interior of a bent right knee joint as seen from the opposite side from that in FIGS. 5A and 5B.

FIG. 7 is a side view in which an ultrasound measurement device has been brought into contact with a bent right knee.

FIG. 8 is a side view of an ultrasound measurement device that has been brought into contact with a bent right knee, on the opposite side from that in FIG. 7.

FIGS. 9A, 9B and 9C are diagrams of the configuration of an ultrasound measurement device in which silicone rubber is provided to the nozzle.

FIG. 10 is a flowchart of a method for measuring knee joint cartilage using an ultrasound measurement device.

### DETAILED DESCRIPTION OF EMBODIMENTS

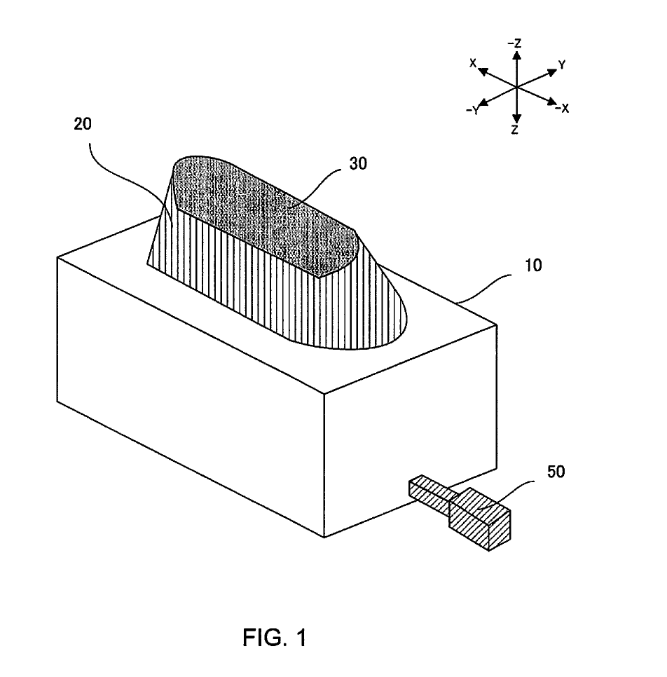

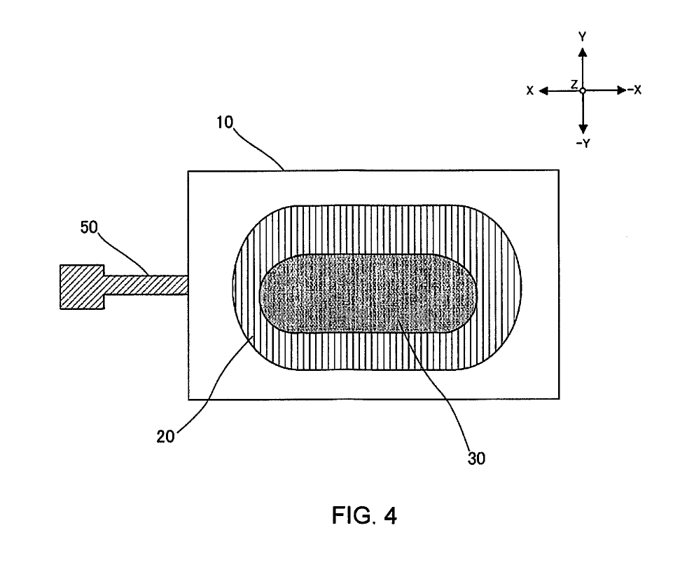

FIGS. 1 to 4 show the exterior and the configuration of the ultrasound measurement device of the present invention. As shown in FIGS. 1 to 4, the ultrasound measurement device is formed from a main body component 10, a nozzle 20, a silicone film 30, an ultrasound probe 40, a drive mechanism 50, and water 60.

The main body component 10 is cuboid in appearance, the bottom face of which is rectangular in shape. The main body component 10 holds the water 60, which transmits ultrasonic waves, and the ultrasound probe 40 in the interior of this hollow shape. The major axis is disposed from the +X direction to the −X direction. In embodiments, the face of the main body component 10 where the nozzle 20 is located will be called the bottom face, the face on the opposite side from the bottom face will be called the top face, and side face in the +Y direction will be called the left side face, the side face in the −Y direction will be called the right side face, the side face in the +X direction will be called the front face, the side face in the −X direction will be called the rear face, the plane composed of the X axis and the Y axis will be called the XY plane, the plane composed of the X axis and the Z axis will be called the XZ plane, and the plane composed of the Y axis and the Z axis will be called the YZ plane. The bottom face and top face are parallel to the XY plane, and the right side face and left side face are parallel to the XZ plane. The interior of the main body component 10 is hollow.

The nozzle 20 has a column shape and a hollow shape in appearance. The nozzle 20 touches the approximate center of the bottom face of the main body component 10. When divided along a plane parallel to the XY plane, the nozzle 20 is composed of two straight parts that are perpendicular to the Y axis, and two curved parts that link the ends of the two straight parts. The two straight parts have the same length. The curves are semicircular arcs that protrude in the +X and −X directions. The length of the outer periphery of a cross section of the nozzle 20 is greatest on the bottom face side of the main body component, and decreases in the −Z direction. Specifically, the nozzle 20 has a shape that is constricted in the −Z direction.

The nozzle 20 communicates with the main body component 10 in the −Z direction via a hollow portion 21. Since the nozzle 20 is integrated with the interior of the main body component 10 by the hollow portion 21, it holds the water 60 integrally with the main body component 10.

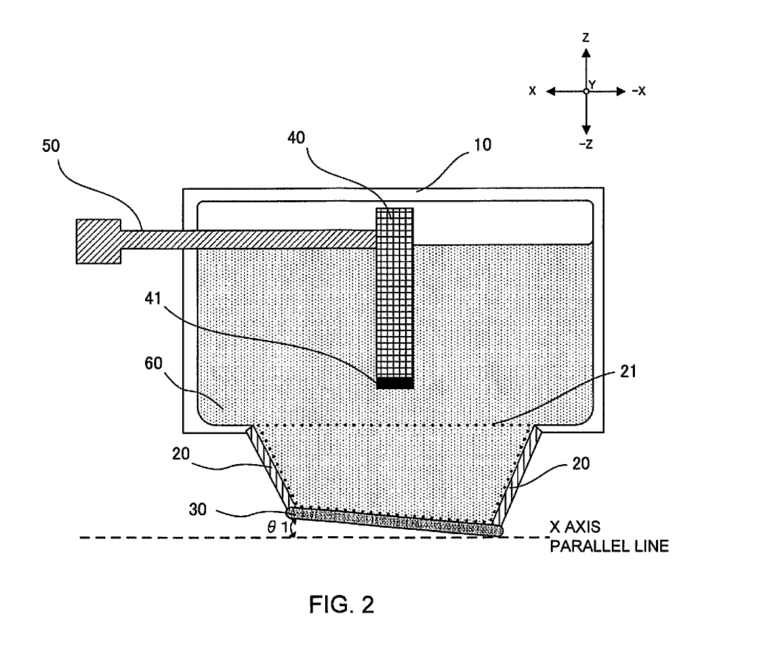

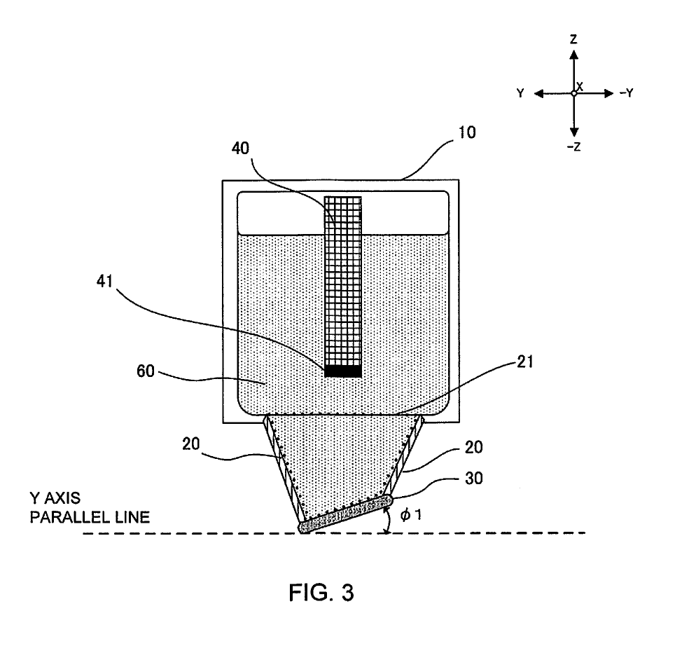

As shown in FIG. 2, the face of the nozzle 20 in the −Z direction is inclined at an angle of θ1 to the X axis, and as shown in FIG. 3, is inclined at an angle of φ1 to the Y axis.

The silicone film 30 has the same planar shape as the face of the nozzle 20 in the −Z direction, and is composed of a material that transmits ultrasonic waves. The silicone film 30 is provided so as to block the face of the nozzle 20 in the −Z direction. Therefore, the water 60 will not leak out from the face of the nozzle 20 in the −Z direction of the main body component 10.

Because the face of the nozzle 20 in the −Z direction is inclined to the X axis and the Y axis, the silicone film 30 is inclined at θ1 to the X axis as shown in FIG. 2, and is inclined at φ1 to the Y axis as shown in FIG. 3.

The ultrasound probe 40 has a circular column shape. The face on the −Z side of the ultrasound probe 40 is parallel to the bottom face of the main body component 10. Also, the main body component 10 is tall enough that the ultrasound probe 40 does not touch the silicone film 30 or the top face in the interior of the main body component 10.

A transducer 41 is provided on the −Z side of the ultrasound probe 40. The transducer 41 is electrically connected to a signal processor (not shown). The transducer 41 sends and receives ultrasonic wave, and outputs electrical signals corresponding to the intensity of the received ultrasonic waves to the signal processor (not shown).

The drive mechanism 50 passes through the rear face of the main body component 10 and is connected to the ultrasound probe 40. The drive mechanism 50 moves the ultrasound probe parallel to the X axis. The ultrasound measurement device is configured so that the place to be measured with ultrasound can be moved along the X axis.

The ultrasonic waves emitted from the transducer 41 advance through the water 60 perpendicularly to the bottom face of the main body component 10, and advance obliquely to the silicone film 30.

Next, the shape of a knee joint will be described in detail.

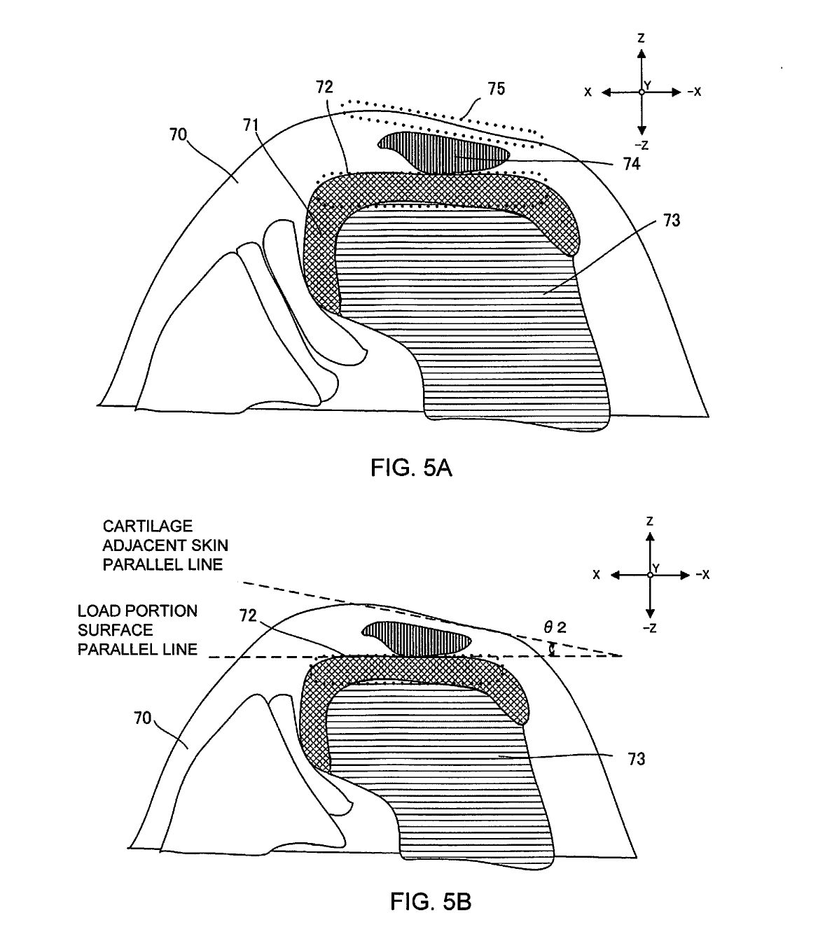

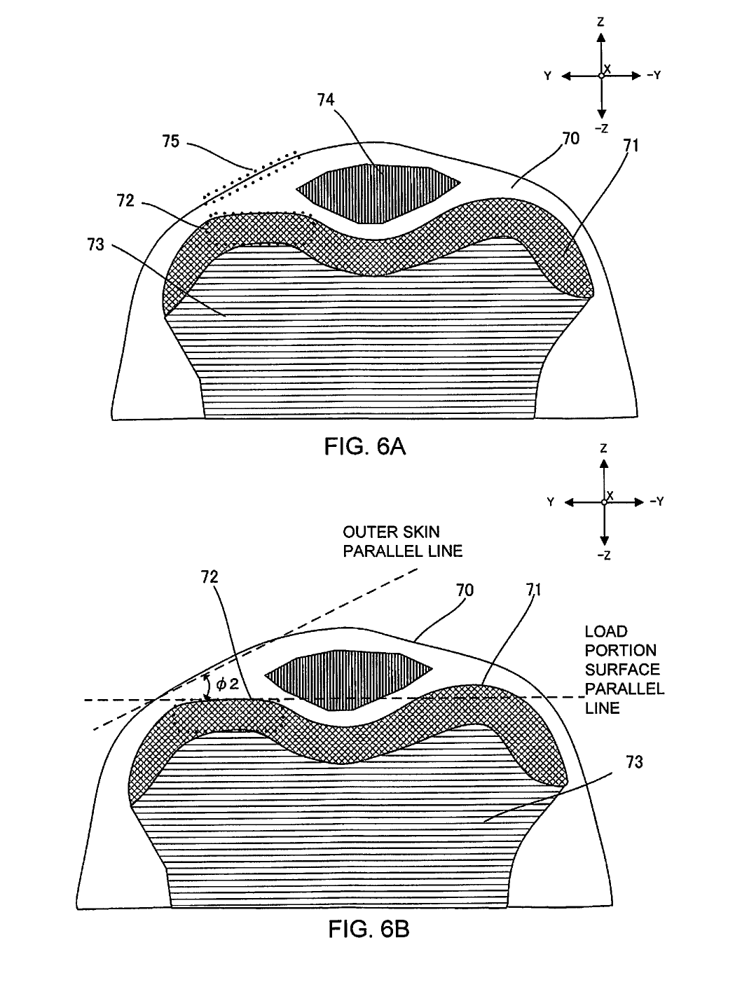

FIGS. 5A and 6A show the interior of a joint when a right knee has been bent at least 90 degrees. FIG. 5A is a cross section of the right knee as seen from the inside out. FIG. 6A is a view of the right knee as seen from the trunk side toward the feet. FIGS. 5B and 6B show the relation between the outer skin and the surface of the cartilage when the right knee is bent at least 90 degrees. In FIGS. 5A and 5B, the +X side is the foot side, and the −X side is the trunk side. In FIGS. 6A and 6B, the +Y side is the inside of the right knee, and the −Y side is the outside of the right knee. In FIGS. 5A, 5B, 6A, and 6B, the +Z side is the front side of the knee, and the −Z side is the back side of the knee.

Of the knee joint cartilage 71 on the foot side of the thigh bone 73, a load portion 72 is the portion subjected to the greatest load. The surface of the load portion 72 is exposed under the skin when the knee is bent, making measurement easier. The cartilage adjacent skin 75 is the portion of the outer skin 70 that is adjacent to the surface of the load portion 72.

The cartilage adjacent skin 75 is inclined with respect to the surface of the load portion 72. More specifically, as shown in FIG. 5B, the cartilage adjacent skin 75 is inclined at an angle θ2 with respect to the surface of the load portion 72 in the XZ plane. As shown in FIG. 6B, the cartilage adjacent skin 75 is inclined at an angle φ2 with respect to the surface of the load portion 72 in the YZ plane. Therefore, it ultrasonic wave should happen to be perpendicularly incident on the cartilage adjacent skin 75, the ultrasonic wave will not be perpendicularly incident on the surface of the load portion 72, and will not be reflected perpendicularly by the surface of the load portion 72.

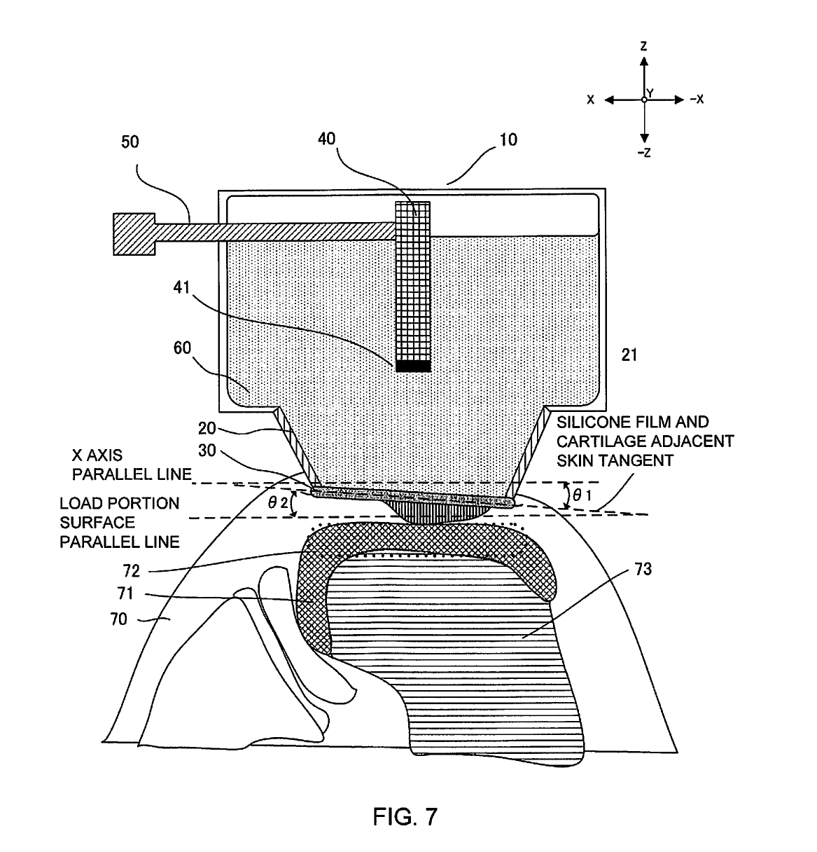

FIG. 7 shows the position of the knee joint and the ultrasound measurement device when the ultrasound measurement device of the present invention is brought into contact with a right knee that has been bent at 120 degrees. FIG. 7 is a view of the right knee as seen from the inside out. In FIG. 7, the +X side is the foot side, while the −X side is the trunk side. The +Z side is the front side of the knee, and the −Z side is the back side of the knee. The silicone film 30 is used by being placed in contact with the cartilage adjacent skin 75 so as to form a single plane, and parallel to the cartilage adjacent skin 75. The bottom face of the main body component 10 is parallel to the XY plane.

The silicone film 30 and the cartilage adjacent skin 75 are inclined at the angle θ1 with respect to the X axis (the bottom face of the main body component 10). The silicone film 30 and the cartilage adjacent skin 75 are inclined at the angle θ2 in the XZ plane with respect to the surface of the load portion 72. Since the angle θ1 is set to be the substantially same as the angle θ2, the surface of the load portion 72 is substantially parallel to the X axis (the bottom face of the main body component 10).

Thus, the user can make the bottom face of the main body component 10 parallel to the surface of the load portion 72 in the XZ plane merely by placing the ultrasound measurement device of the present invention against the cartilage adjacent skin 75.

The ultrasonic waves emitted from the transducer 41 are transmitted through the silicone film 30, the cartilage adjacent skin 75, and the soft tissue, in that order, and then reach the load portion 72. In the XZ plane, the angle at which the ultrasonic waves advance is perpendicular to the surface of the load portion 72 because the surface of the load portion 72 and the bottom face of the main body component 10 are parallel.

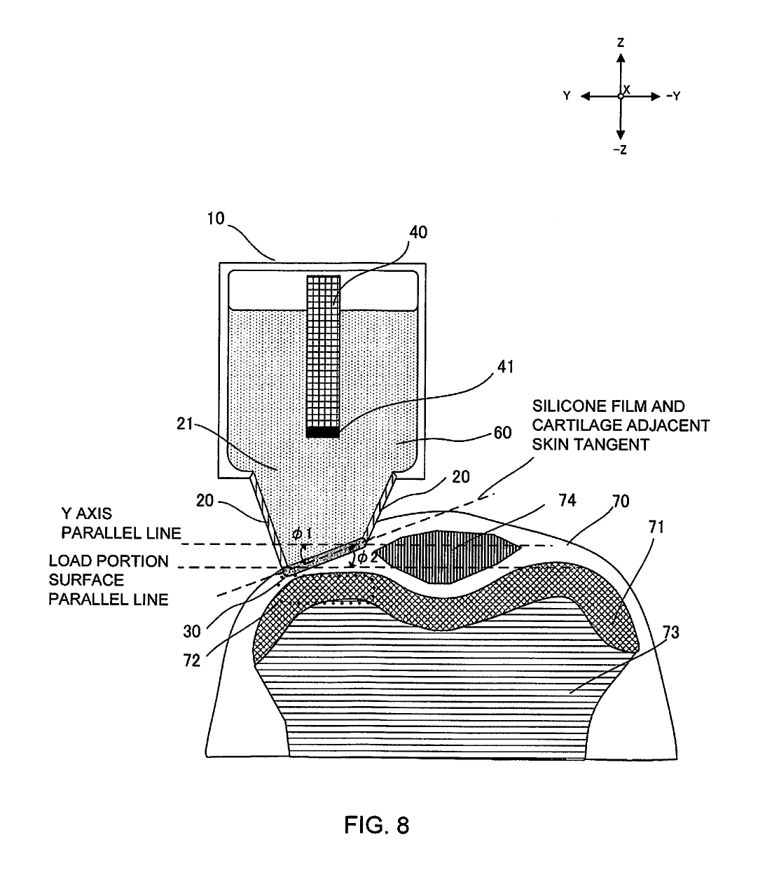

FIG. 8 is a view of the ultrasound measurement device and the right knee shown in FIG. 7 as seen from the trunk side toward the foot side of the patient. The −Y side is the outside of the right knee, while the +Y side is the inside of the right knee.

The bottom face of the main body component 10 and the silicone film 30 and cartilage adjacent skin 75 are inclined at the angle φ1 with respect to the Y axis (the bottom face of the main body component 10). The silicone film 30 and the cartilage adjacent skin 75 are inclined at the φ2 in the YZ plane with respect to the surface of the load portion 72. Since the angle φ1 is set to be substantially the same as the angle φ2, the surface of the load portion 72 is substantially parallel to the Y axis (the bottom face of the main body component 10).

Thus, the user can make the bottom face of the main body component 10 parallel to the surface of the load portion 72 in the YZ plane merely by placing the ultrasound measurement device of the present invention against the cartilage adjacent skin 75.

The ultrasonic waves emitted from the transducer 41 advance perpendicular to the surface of the load portion 72 because the bottom face of the main body component 10 and the surface of the load portion 72 are parallel in the YZ plane.

As discussed above, with the ultrasound measurement device of the present invention, the angle at which the ultrasonic waves advance can be made perpendicular to the surface of the load portion 72 of the knee cartilage. Since the acoustic impedance in soft tissue is different from the acoustic impedance in the knee joint cartilage 71, the ultrasonic waves that reach the surface of the load portion 72 are reflected by the surface of the load portion 72. The reflected waves face the opposite direction from the direction in which the ultrasonic waves come out of the transducer 41. Therefore, the reflected waves reach the transducer 41 in the shortest distance from the surface of the load portion 72.

The angle at which the ultrasonic waves advance with respect to the surface of the load portion 72 in FIG. 7 will be perpendicular as long as the surface of the load portion 72 is flat, even if the ultrasound probe 40 is moved along the X axis by the drive mechanism 50. Therefore, the ultrasound measurement device of the present invention can measure ultrasonic waves by using the drive mechanism 50 to continuously move the surface of the load portion 72 in parallel along the X axis.

The angles θ1 and φ1 of the ultrasound measurement device of the present invention preferably match up perfectly with the angles θ2 and φ2. However, the angles θ2 and φ2 may have some variance, depending on the patient. The user will need to make fine adjustments to the angle of the ultrasound probe 40 when measuring a patient in which the angles θ2 and φ2 vary greatly in proportion to the average values for the patient.

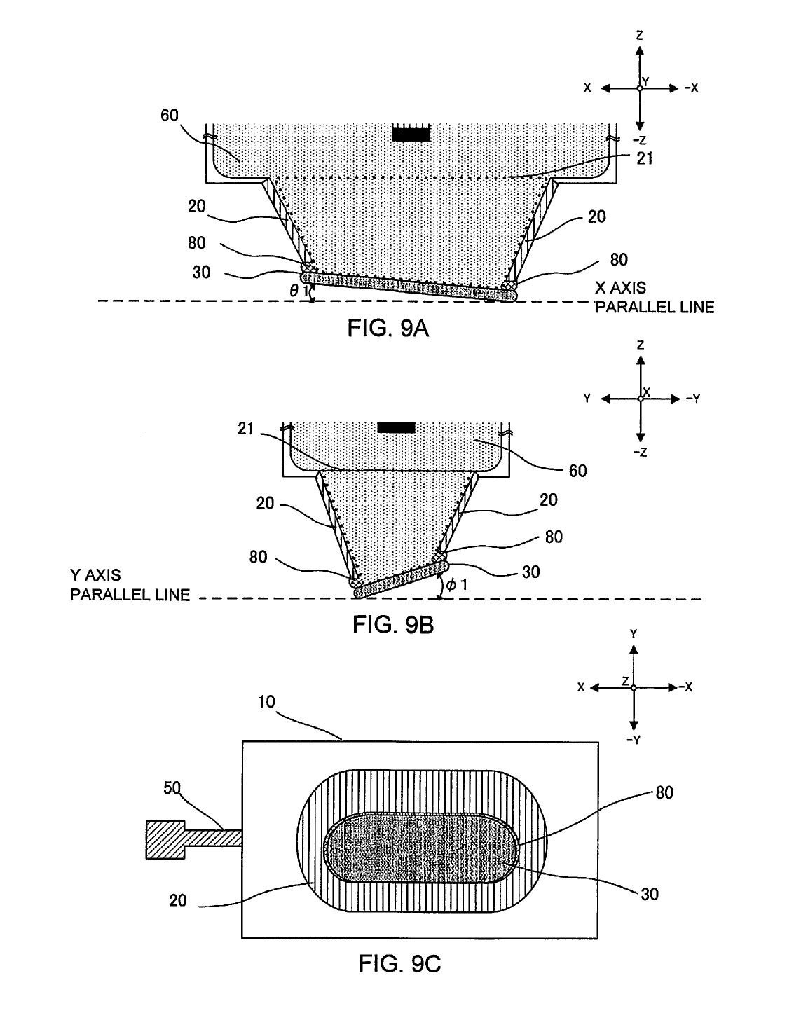

In view of this, in addition to the ultrasound measurement device shown in FIGS. 1 to 4, an ultrasound measurement device that further comprises a silicone rubber piece 80 as an elastic body will now be discussed. In FIG. 9, those components that are the same as in FIGS. 1 to 4 will not be described again.

FIG. 9A is a cross section along the XY plane of the ultrasound measurement device equipped with the silicone rubber piece 80, FIG. 9B is a cross section along the YZ plane, and FIG. 9C is a bottom view. The silicone rubber piece 80 is provided between the silicone film 30 and the portion of the nozzle 20 that is in contact with the silicone film 30. As shown in FIG. 9C, the silicone rubber piece 80 has the same shape as the cross sectional view of the nozzle 20. The silicone rubber piece 80 has a ring shape that goes around the edge of the nozzle 20 and is joined on the −Z side of the edge of the nozzle 20. The silicone film 30 is joined to the silicone rubber piece 80 on the opposite side (−Z side) of the silicone rubber piece 80 from the edge of the nozzle 20.

The silicone rubber piece 80 deforms when integrally pressed against the ultrasound measurement device by the user when the silicone film 30 is brought into contact with the cartilage adjacent skin 75. The extent to which the silicone rubber piece 80 deforms varies with an adjustment in which the user increases the force pressing on the +X side over the force pressing on the −X side, for example. In FIG. 9A, for instance, if the pressing force in the −Z direction on the −X side of the silicone rubber piece 80 is increased over the pressing force in the −Z direction on the +X side, the −X side is compressed more along the Z axis than the portion on the +X side. The angle θ1 then becomes greater than before the pressing force was increased. Thus, the user can make fine adjustments to the angles θ1 and φ1 by pressing on and deforming the silicone rubber piece 80. Also, if the nozzle 20 is made of metal, because the silicone rubber piece 80 is compressed when pressed against the patient, the local pressure on the biological surface is dispersed, which reduces the damage to the biological surface and alleviates the burden on the patient.

As discussed above, with the ultrasound measurement device of the present invention, which comprises the silicone rubber piece 80, the angles θ1 and φ1 can be finely adjusted to match the shape of the patient's knee joint, and even though the angles θ2 and φ2 vary depending on the patient, the angle at which the ultrasonic waves advance can be made perpendicular to the surface of the load portion 72.

In the above example, the main body component 10 and the nozzle 20 are configured integrally, but the configuration may allow the nozzle 20 to be replaced. If the nozzle 20 and the silicone film 30 are replaced, then threaded holes are made in the bottom face of the main body component 10, for example, and the nozzle 20 and the silicone film 30 are attached to the main body component 10 with screws. The user can join the nozzle 20 and the main body component 10 together with screws and thereby attach the nozzle 20 and the silicone film 30 to the main body component 10. Therefore, the user can replace the nozzle 20 to match the shape of the patient's knee joint, and can make the advance angle of the ultrasonic waves perpendicular to the surface of the load portion 72.

In the above example, the face of the nozzle 20 in the −Z direction had a flat shape composed of two straight parts and two curved parts, but this shape is not the only option. For example, the nozzle 20 can be a three-dimensional shape that matches the three-dimensional shape of the patient's knee, which will allow the angle in which the ultrasonic waves advance to be made perpendicular to the surface of the load portion 72 more accurately.

The drive mechanism 50 moves the ultrasound probe 40 parallel to the X axis, but the direction is not limited to the X axis. The drive mechanism 50 can also move the ultrasound probe 40 parallel to the Y axis. In this case, the ultrasound measurement device can continuously measure the flat shape of the surface of the load portion 72. Furthermore, the drive mechanism 50 can move in the Z axis direction. In this case, even if the soft tissue is thick and the cartilage adjacent skin 75 is farther away from the surface of the load portion 72, the user can still move the probe in the Z axis direction and thereby adjust the focal depth of the emitted ultrasonic waves, so that the ultrasonic waves are emitted properly.

In the above example, scanning by mechanical drive was used, but it is also possible for numerous transducer elements to be arranged in an array. With the ultrasound measurement device of the present invention, if the ultrasound probe is configured with numerous transducer elements arranged in an array, then ultrasonic waves can be sent and received all at once even without successive scanning by mechanical drive. Therefore, the user can measure cartilage in a knee joint in a short time, which means that the patient does not have to suffer through a prolonged measurement.

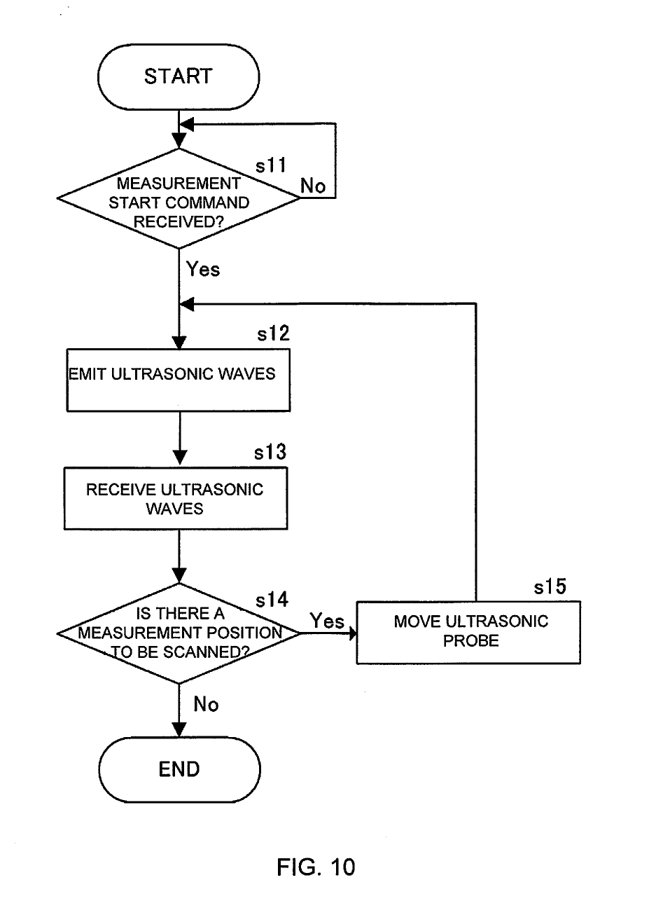

FIG. 10 is a flowchart of a method for measuring knee joint cartilage using the ultrasound measurement device of the present invention. When the ultrasound measurement device receives a command to start measurement (S11), the ultrasound probe 40 emits ultrasonic waves toward the cartilage in the knee joint (S12). The ultrasound probe 40 then receives the ultrasonic waves reflected by the load portion 72 (S13). When ultrasonic waves are sent and received at other measurement positions by scanning (Yes in S14), the drive mechanism 50 moves the ultrasound probe 40 (S15). The flow then returns to step S12, and measurements are successively made at different positions. Once measurement is finished at all of the measurement positions (No in S14), the ultrasound measurement is ended.

As discussed above, the ultrasound measurement method of the present invention allows cartilage to be properly measured by having ultrasonic waves be perpendicularly incident on the surface of the load portion 72 of the knee joint cartilage 71, and successively scanning.

1. An ultrasound measurement instrument comprising:
a main body component;
a nozzle provided in one plane of the main body component, the nozzle having a hollow shape and communicating with the main body component via a hollow portion; and
a contact component blocking off a face of the nozzle on the opposite side from the main body component in a flat shape such that ultrasonic waves are transmitted,
the contact component being inclined with respect to a first axis that is parallel to the plane of a hollow body in which the nozzle is provided, and to a second axis that is perpendicular to the first axis and is parallel to the plane.

2. The ultrasound measurement instrument according to claim 1, wherein
the main body component has a polyhedral shape.

3. The ultrasound measurement instrument according to claim 1, wherein
the main body component has a hollow sphere shape.

4. The ultrasound measurement instrument according to claim 1, wherein
an angle at which the contact component is inclined with respect to a face of the main body component where the nozzle is provided corresponds to an angle at which a surface of knee joint cartilage is inclined with respect to an outer skin surface of a knee joint.

5. The ultrasound measurement instrument according to claim 1, wherein
an elastic body is provided between the contact component and a portion of the nozzle that touches the contact component.

6. The ultrasound measurement instrument according to claim 5, wherein
the elastic body is silicone rubber.

7. The ultrasound measurement instrument according to claim 1, wherein
the nozzle has a column shape that is constricted toward a face that is opposite a face of the main body component where the nozzle is provided.

8. The ultrasound measurement instrument according to claim 1, wherein
the contact component has a shape that combines a straight part and a curved part.

9. The ultrasound measurement instrument according to claim 1, wherein
the contact component is a silicone film.

10. An ultrasound measurement device comprising:
the ultrasound measurement instrument according to claim 1;
an ultrasound probe configured to transmit and receive ultrasonic waves that pass through the contact component; and
a drive mechanism configured to drive the ultrasound probe in an interior of the main body component.

11. The ultrasound measurement device according to claim 10, wherein
the drive mechanism moves the ultrasound probe parallel to a face of the main body component where the nozzle is provided.

12. The ultrasound measurement device according to claim 11, wherein
the drive mechanism moves the ultrasound probe perpendicular to a face of the main body component where the nozzle is provided.

13. A method for ultrasonically measuring knee joint cartilage comprising:
communicating with a contact component that is inclined with respect to a first axis that is parallel to plane of a hollow body in which a nozzle is provided, and to a second axis that is perpendicular to the first axis and is parallel to the plane;
emitting ultrasonic waves perpendicular to the plane; and
receiving ultrasonic waves that have been reflected by knee joint cartilage and have passed through the contact component.

14. The ultrasound measurement instrument according to claim 2, wherein
an angle at which the contact component is inclined with respect to a face of the main body component where the nozzle is provided corresponds to an angle at which a surface of knee joint cartilage is inclined with respect to an outer skin surface of a knee joint.

15. The ultrasound measurement instrument according to claim 3, wherein
an angle at which the contact component is inclined with respect to a face of the main body component where the nozzle is provided corresponds to an angle at which a surface of knee joint cartilage is inclined with respect to an outer skin surface of a knee joint.

16. The ultrasound measurement instrument according to claim 2, wherein
an elastic body is provided between the contact component and a portion of the nozzle that touches the contact component.

17. The ultrasound measurement instrument according to claim 3, wherein
an elastic body is provided between the contact component and a portion of the nozzle that touches the contact component.

18. The ultrasound measurement instrument according to claim 4, wherein
an elastic body is provided between the contact component and a portion of the nozzle that touches the contact component.

19. The ultrasound measurement instrument according to claim 2, wherein
the nozzle has a column shape that is constricted toward a face that is opposite a face of the main body component where the nozzle is provided.

20. The ultrasound measurement instrument according to claim 3, wherein
the nozzle has a column shape that is constricted toward a face that is opposite a face of the main body component where the nozzle is provided.
